(19) United States
(12) Patent Application Publication
(10) Pub. No.: US 2021/0393425 A1
(43) Pub. Date: Dec. 23, 2021

(54) STENT DELIVERY SYSTEM

(71) Applicant: ASAHI INTECC CO., LTD., Seto-shi (JP)

(72) Inventors: Yukihiro FUSEYA, Seto-shi (JP); Marina TSUZUKU, Seto-shi (JP)

(73) Assignee: ASAHI INTECC CO., LTD., Seto-shi (JP)

(21) Appl. No.: 17/465,306

(22) Filed: Sep. 2, 2021

Related U.S. Application Data
(63) Continuation of application No. PCT/JP2020/003348, filed on Jan. 30, 2020.

(30) Foreign Application Priority Data
Mar. 5, 2019 (JP) 2019-039575

(51) Int. Cl. A61F 2/966

(52) U.S. Cl. CPC A61F 2/966; A61M 25/0069

(57) ABSTRACT

A stent delivery system includes a distal end shaft having a tapered portion, a spirally-arranged protruding portion provided on an outer peripheral face of the tapered portion, an inner shaft extending toward a proximal end side in a longitudinal axis direction of the distal end shaft, a self-expanding stent, and an outer shaft. The outer shaft is slidable between a first position where the stent in a contracted state is covered and a second position on the proximal end side of the stent in the longitudinal axis direction of the inner shaft. An outermost periphery of the outer shaft fits inside an outermost periphery of the spirally-arranged protruding portion in a frontal view from the distal end side of the distal end shaft in the longitudinal axis direction.

### CROSS REFERENCE TO RELATED APPLICATION

This application is a continuation application of International Application No. PCT/JP2020/003348, filed Jan. 30, 2020, which claims priority to Japanese Patent Application No. 2019-039575, filed Mar. 5, 2019. The contents of these applications are incorporated herein by reference in their entirety.

### TECHNICAL FIELD

The present disclosure relates to a stent delivery system.

### BACKGROUND

As a device for widening a penetration-pore or a constricted part of a blood vessel, a digestive tract, or the like caused by a lesion or the like, for example, a stent obtained by weaving a metal wire in a mesh shape is known.

When widening a constricted part caused in a blood vessel, a digestive tract, or the like using such a stent, it is necessary to preliminarily widen the constricted part to a size allowing insertion of the stent in a diameter-decreased state prior to placement of the stent in the constricted part. As an instrument used for such preliminary manipulation, for example, dilators have been proposed.

A dilator has a tapered distal end portion that increases in diameter toward a proximal end side, and when the tapered part passes through a constricted part or the like, this constricted part or the like is widened according to JP2008-11867. Then, the stent in a diameter-decreased state is inserted into the widened constricted part or the like, then this stent is widened outward in a radial direction and placed in the constricted part, so that the constricted part of the blood vessel, the digestive tract, or the like can be stably widened.

However, when using the conventional dilator as described above, it is necessary to transport the stent to the constricted part or the like after drawing the dilator out of the preliminarily widened constricted part or the like. Thus, the preliminarily widened constricted part or the like may be constricted again in a body cavity such as a significantly elastic blood vessel or digestive tract, complicating the procedure by requiring additional preliminary manipulation, or the like.

### SUMMARY

The present disclosure was made based on the above circumstances, and an object of the disclosed embodiments is to provide a stent delivery system capable of smoothly placing a stent in a body cavity such as a blood vessel and a digestive tract.

To achieve the above object, a stent delivery system according to an embodiment of the present disclosure includes a distal end shaft that has a through-hole, and a tapered portion having an outer diameter gradually increasing from a distal end to a proximal end; a spirally-arranged protruding portion that is provided on an outer peripheral face of the tapered portion and has a gap between adjacent sections along a longitudinal axis direction of the distal end shaft (also simply referred to as “longitudinal direction”); an inner shaft that is connected to the proximal end of the distal end shaft, has a through-hole communicating with the through-hole of the distal end shaft, and extends toward the proximal end side in the longitudinal axis direction of the distal end shaft; a self-expanding stent that covers the inner shaft and is expandable and contractable in a radial direction of the inner shaft; and an outer shaft that covers the inner shaft and is slidable along a longitudinal axis direction of the inner shaft, wherein the outer shaft is slidable between a first position where the stent in a contracted state is covered by the outer shaft and a second position on the proximal end side of the stent in the longitudinal axis direction of the inner shaft, and an outermost periphery of the outer shaft fits inside an outermost periphery of the spirally-arranged protruding portion in a frontal view from the distal end side of the distal end shaft in the longitudinal axis direction.

### BRIEF DESCRIPTION OF THE DRAWINGS

FIG. 1 is a schematic side view of an embodiment of the disclosed embodiments, illustrating a state in which an outer shaft is at a first position;

FIG. 2 is a schematic front view of the embodiment in FIG. 1;

FIG. 3 is a schematic side view of an embodiment of the disclosed embodiments, illustrating a state in which an outer shaft is at a second position;

FIG. 4A is a schematic side view illustrating an example of using the device of the embodiment in FIG. 1;

FIG. 4B is a schematic side view illustrating an example of using the device of the embodiment in FIG. 1;

FIG. 4C is a schematic side view illustrating an example of using the device of the embodiment in FIG. 1;

FIG. 4D is a schematic side view illustrating an example of using the device of the embodiment in FIG. 1;

FIG. 4E is a schematic side view illustrating an example of using the device of the embodiment in FIG. 1;

FIG. 4F is a schematic side view illustrating an example of a using the device of the embodiment in FIG. 1;

FIG. 5A is a schematic side view of an embodiment of the disclosed embodiments, illustrating a state in which the outer shaft is at the first position;

FIG. 5B is a schematic side view of an embodiment of the disclosed embodiments, illustrating a state in which the outer shaft is at the first position; and

FIG. 5C is a schematic side view of an embodiment of the disclosed embodiments, illustrating a state in which the outer shaft is at the first position.

### DETAILED DESCRIPTION

The stent delivery system includes a distal end shaft that has a through-hole and a tapered portion having an outer diameter gradually increasing from a distal end to a proximal end; a spirally-arranged protruding portion that is provided on an outer peripheral face of the tapered portion and has a gap between adjacent sections along a longitudinal axis direction of the distal end shaft; an inner shaft that is connected to the proximal end of the distal end shaft and has a through-hole communicating with the through-hole of the distal end shaft, and extends toward the proximal end side of the distal end shaft in the longitudinal axis direction; a self-expanding stent that covers the inner shaft and is expandable and contractable in a radial direction of the inner shaft; and an outer shaft that covers the inner shaft and is slidable along a longitudinal axis direction of the inner shaft. The outer shaft is slidable between a first position where the stent in a contracted state is covered by the outer shaft and a second position on the proximal end side of the stent in the longitudinal axis direction of the inner shaft. An outermost periphery of the outer shaft fits inside an outermost periphery of the spirally-arranged protruding portion in a frontal view from the distal end side of the distal end shaft in the longitudinal axis direction.

Note that in the present disclosure, the “distal end side” means a direction along the longitudinal axis direction of the distal end shaft, in which the distal end shaft is located with respect to the inner shaft. The “proximal end side” means a direction along the longitudinal axis direction of the distal end shaft, which is a direction opposite to the distal end side. The “distal end” refers to a distal end portion of any member or part, and the “proximal end” refers to a proximal end portion of any member or part. The “outermost periphery” means a shape formed by an outline of a particular member (spirally-arranged protruding portion, outer shaft, etc.) as viewed from the longitudinal axis direction of the distal end shaft.

Hereinafter, an embodiment of the disclosed embodiments will be explained with reference to the figures, but the disclosed embodiments are not limited only to the embodiments illustrated in the figures. Note that the dimensions of the stent delivery system indicated in the figures are illustrated for the purpose of facilitating understanding of the implementation details, and do not necessarily correspond to the actual dimensions.

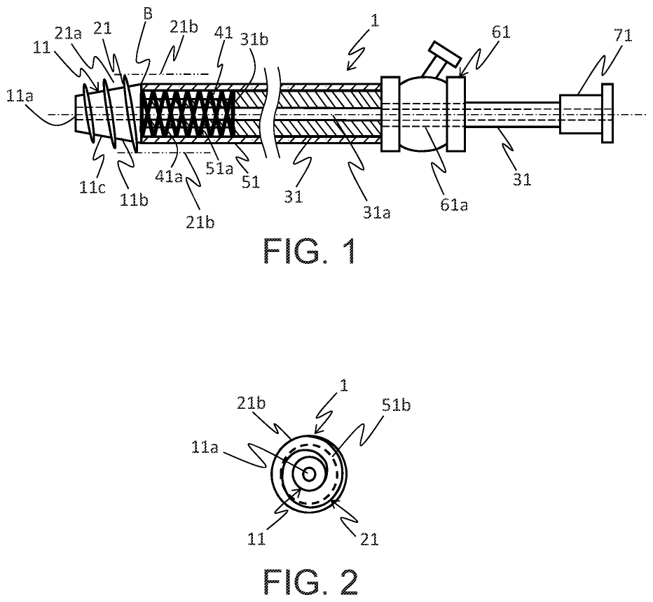

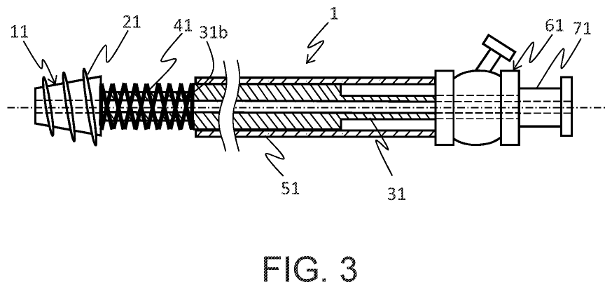

FIG. 1 is a schematic side view of an embodiment of the disclosed embodiments, illustrating a state in which the outer shaft is at the first position. FIG. 2 is a schematic front view of the embodiment in FIG. 1. As illustrated in FIG. 1 and FIG. 2, a stent delivery system 1 generally includes a distal end shaft 11, a spirally-arranged protruding portion 21, an inner shaft 31, a stent 41, an outer shaft 51, and a connector 61.

The distal end shaft 11 has a through-hole 11a, and a tapered portion 11b having an outer diameter gradually increasing from a distal end to a proximal end. Specifically, the through-hole 11a can be composed of e.g. a through-hole passing from the distal end to the proximal end of the distal end shaft 11. As the tapered portion 11b, it is possible to adopt a tapered portion that has e.g. an outer periphery shape gradually increasing in diameter from the distal end to the proximal end (see FIG. 1), a tapered portion having a diameter stepwisely increasing from the distal end to the proximal end (not illustrated), and a combination thereof (not illustrated), or the like. For example, a guide wire (not illustrated) may be inserted into the through-hole 11a, and the stent delivery system 1 advances and retreats along the guide wire.

A material constituting the distal end shaft 11 preferably has antithrombogenicity, flexibility, and biocompatibility because the shaft is inserted into a body cavity. For example, it is possible to adopt a resin material such as a polyamide resin, a polyolefin resin, a polyester resin, a polyurethane resin, a silicone resin, and a fluororesin; or a metal material such as a stainless steel and a superelastic alloy (nickel-titanium alloy), or the like.

The distal end shaft 11 may have various coating films (not illustrated) on a side of an outer peripheral face 11c of the distal end shaft 11. Examples of such coating films include a protective film (typified by a plating film) for protecting the surface of the distal end shaft 11, a base film for improving adhesiveness between the distal end shaft 11 and a spirally-arranged protruding portion 21 described later, and the like.

The spirally-arranged protruding portion 21 is provided on the outer peripheral face 11c of the tapered portion 11b and has a gap 21a between adjacent sections along the longitudinal axis direction of the distal end shaft 11 (adjacent protruding portions of the spirally-arranged protruding portion 21 are separated in the longitudinal axis direction). Specifically, for example, this spirally-arranged protruding portion 21 protrudes from the outer peripheral face 11c of the distal end shaft 11 outward in a radial direction of the distal end shaft 11, and is formed such that the outermost periphery of the spirally-arranged protruding portion 21 (see the two-dot chain line 21b in FIG. 1) is located on the radial outside of the outermost periphery of the distal end shaft (in the stent delivery system 1, the outer shape of the proximal end of the distal end shaft 11) in a frontal view from the distal end side in the longitudinal axis direction of the stent delivery system 1.

The spirally-arranged protruding portion 21 can be formed as a continuous or intermittent single-thread or multi-thread protruding portion. In addition, the spirally-arranged protruding portion 21 can also be formed by spirally winding one or more wires around the outer peripheral face 11c of the distal end shaft 11. The spirally-arranged protruding portion 21 may be either integrated with or separated from the distal end shaft 11. In the stent delivery system 1, the spirally-arranged protruding portion 21 is formed as a continuous single-thread protruding portion so as to be integrated with the distal end shaft 11 by casting or the like.

A part where the spirally-arranged protruding portion 21 is formed on the distal end shaft 11 may be any part between the distal end and the proximal end of the distal end shaft 11 in the longitudinal axis direction. For example, it is possible to adopt a configuration in which the spirally-arranged protruding portion is formed entirely from the distal end to the proximal end of the distal end shaft 11 (see FIG. 1), a configuration in which the spirally-arranged protruding portion is formed from the distal end to a middle of the distal end shaft (not illustrated), a configuration in which the spirally-arranged protruding portion is formed from the middle to the proximal end of the distal end shaft (not illustrated), or the like.

Preferably, the spirally-arranged protruding portion 21 does not constitute a blade (it is shaped so as to not cut biological tissues). That means, the spirally-arranged protruding portion 21 preferably has a transverse section (section orthogonal to the spiral direction of the spirally-arranged protruding portion 21) in which an outside end portion (apex portion) of the spirally-arranged protruding portion 21 in the radial direction of the distal end shaft 11 is not an acute corner portion. Examples of such an end portion include an end portion composed of an obtuse corner, an end portion composed of a corner portion shaped so as to include a curve (e.g. a curve including a part of a circle or an ellipse, etc.), and the like. Thereby, the stent delivery system 1 can widen a hole of an object without damaging biological tissues on an inner wall face of the hole of the object when performing a preliminary operation (preliminary widening of a constricted part or the like before inserting a diameter-decreased stent).

When the spirally-arranged protruding portion and the distal end shaft are separately formed, for example, the same material as the material constituting the aforementioned distal end shaft, or the like can be adopted as a material constituting the spirally-arranged protruding portion 21.

The inner shaft 31 is connected to the proximal end of the distal end shaft 11, has an through-hole 31a communicating with the through-hole 11a of the distal end shaft 11, and extends toward the proximal end side of the distal end shaft 11 in the longitudinal axis direction. Specifically, the through-hole 31a of the inner shaft 31 is composed of e.g. a through-hole which passes from the distal end to the proximal end of the inner shaft 31. The outer peripheral face of the inner shaft 31 has a hollow cylindrical face on at least a part in the longitudinal axis direction so as to hold the stent 41 (described later). The outer peripheral face of the inner shaft 31 may have a lock portion 31b for locking the proximal end of the stent 41. A proximal end-side part of the outer peripheral face of the inner shaft 31 can be formed in a shape suited to an inner peripheral face of an through-hole 61a of the connector 61 (described later) so as to hold the connector 61 slidably. A handle 71 for rotating the distal end shaft 11 via the inner shaft 31 is connected to the proximal end of the inner shaft 31. The inner shaft 31 and the proximal end of the distal end shaft 11 can be connected to each other, and the proximal end of the inner shaft 31 and the handle 71 can be connected to each other, e.g. by jointing using welding, adhesion using an adhesive, or the like.

As a material constituting the inner shaft 31, for example, the same material as the material constituting the aforementioned distal end shaft 11, or the like can be adopted, because the inner shaft 31 is inserted into the body cavity.

The stent 41 is a self-expanding member that covers the inner shaft 31 and is expandable and contractable in the radial direction of the inner shaft 31. Specifically, as this stent 41, for example, a stent woven into a mesh shape using one or a plurality of metal wires 41a (see FIG. 1), a stent formed into a mesh shape by cutting a part of a substantially-hollow cylindrical body or boring holes thereto using laser processing or the like (not illustrated), or the like can be adopted.

As a material constituting the stent 41, for example, the same material as the material constituting the aforementioned distal end shaft 11, or the like can be adopted, because the stent 41 is placed in the body cavity.

The outer shaft 51 covers the inner shaft 31 and is slidable along the longitudinal axis direction of the inner shaft 31. Specifically, the outer shaft 51 can be composed of e.g. a hollow cylindrical member having a through-hole formed from the distal end to the proximal end such that the stent 41 is wrapped (supported) by an inner peripheral face 51a of the outer shaft 51 and the outer shaft 51 can slide along the longitudinal axis direction of the inner shaft 31.

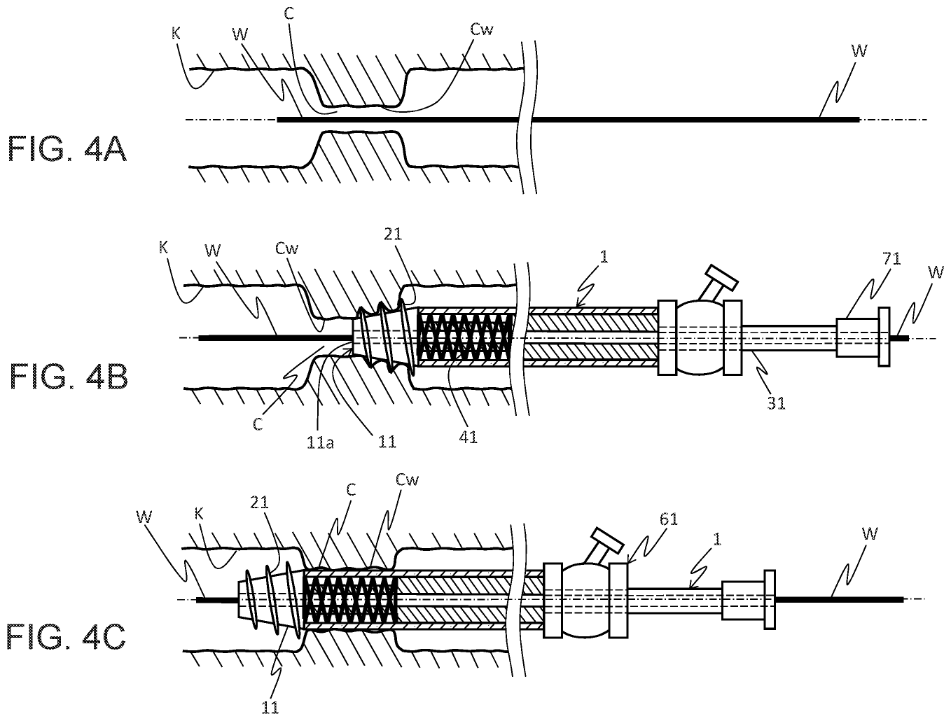

The outer shaft 51 slides in the longitudinal axis direction of the inner shaft 31 between the first position where the stent 41 in a contracted state is covered and the second position on the proximal end side of the stent 41. The first position refers to a position where the entire stent 41 in the contracted state (non-expanded state) is housed inside the outer shaft 51 (see FIG. 1), and the second position refers to a position where the outer shaft 51 is located on the proximal end side of the first position and the stent 41 is completely exposed to the outside (see FIG. 3). The outer shaft 51 can slide e.g. by connecting the proximal end of the outer shaft 51 to the connector 61 and moving the connector 61 forward and backward along the longitudinal axis direction.

An outermost periphery 51b (having the same distal outer shape as of the outer shaft 51, in the stent delivery system 1) of the outer shaft 51 is arranged to fit inside the outermost periphery (see the two-dot chain line 21b in FIG. 1) of the spirally-arranged protruding portion 21 in a frontal view from the distal end side in the longitudinal axis direction of the distal end shaft 11.

Herein, it is possible that the outer diameter of the proximal end of the distal end shaft 11 and the outer diameter of the distal end of the outer shaft 51 substantially coincide with each other, and the proximal end of the distal end shaft 11 and the distal end of the outer shaft 51 contact with each other such that an outer peripheral edge of the proximal end of the distal end shaft 11 and an outer peripheral edge of the distal end of the outer shaft 51 coincide with each other at the first position (see FIG. 1). Thereby, for example, when a boundary portion B between the distal end shaft 11 and the outer shaft 51 passes through a constricted part or the like, the outer shaft 51 can be advanced while the boundary portion B is not caught by an inner wall of the constricted part or the like, resulting in smooth placement of the stent 41. Herein, the outer diameter of the proximal end of the distal end shaft 11 and the outer diameter of the distal end of the outer shaft 51 “substantially coincide” with each other if the outer diameters are sufficiently similar to effectively prevent the boundary portion B from being caught by an inner wall of the constricted part or the like.

As a material constituting the outer shaft 51, for example, the same material as the material constituting the aforementioned distal end shaft 11, or the like can be adopted, because the outer shaft 51 is inserted into the body cavity.

The connector 61 is a part at which an operator operates the stent delivery system 1. This connector 61 has e.g. the through-hole 61a along the longitudinal axis direction and can be configured such that the inner shaft 31 penetrates this through-hole 61a. The outer shape of the connector 61 is not particularly limited as long as the effects of the stent delivery system 1 are not impaired. The connector 61 can be connected to e.g. the proximal end of the outer shaft 51 by jointing using welding, adhesion using an adhesive, or the like.

As for the dimensions of each portion in the stent delivery system 1, in the distal end shaft 11, outer diameters may be from 0.8 to 3.0 mm on the distal end and from 1.4 to 5.0 mm on the proximal end. The spirally-arranged protruding portion 21 may have a maximum diameter of 1.6 to 6.0 mm on the outermost periphery. The outer shaft 51 may have an outer diameter of 1.4 to 6.0 mm. The stent 41 in a contracted state may have an outer diameter of 0.7 to 2.0 mm and a length of 50 to 250 mm. The through-holes 11a and 31a of the distal end shaft 11 and the inner shaft 31 may have an inner diameter of 0.5 to 1.5 mm respectively.

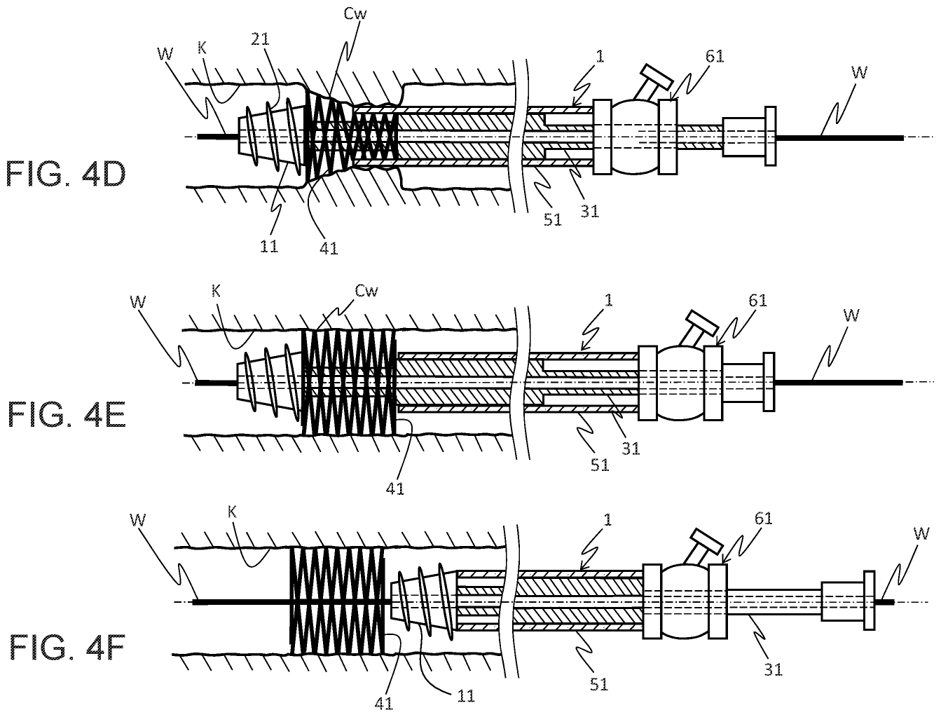

Next, an example of how to use the stent delivery system 1 will be explained with reference to FIG. 4A to FIG. 4F. Herein, an example of a procedure will be explained in which a constricted part C caused in a digestive tract K such as a bile duct and a pancreatic duct due to a lesion is preliminarily widened, and then the stent 41 is placed in the constricted part C.

First, a guide wire W is inserted into the constricted part C prior to insertion of the stent delivery system 1 (see FIG. 4A).

Next, the proximal end of the guide wire W is inserted into the through-hole 11a of the stent delivery system 1 so as to protrude from the proximal end of the stent delivery system 1, and then this stent delivery system 1 is pushed forward to the immediate vicinity of the constricted part C along the guide wire W. Subsequently, the distal end shaft 11 is inserted into the constricted part C from the distal end thereof, then handle 71 is rotated to advance the stent delivery system 1 while the spirally-arranged protruding portion 21 is screwed into an inner wall Cw of the constricted part C (see FIG. 4B), and the advancement of the stent delivery system 1 is stopped at a position where the stent 41 enters the constricted part C (see FIG. 4C). Thereby, the preliminary widening of the constricted part C is completed.

Subsequently, the stent 41 of the stent delivery system 1 is separated from the other part of the stent delivery system 1 to widen the preliminarily widened constricted part C. Specifically, the outer shaft 51 is moved (slid) from the first position to the second position by pulling the connector 61 toward the proximal end side with respect to the inner shaft 31. The stent 41 is gradually exposed as the outer shaft 51 is moved from the first position to the second position. The gradually exposed part of the stent 41 expands outward in the radial direction owing to the self-expanding action of the stent 41 (see FIG. 4D). In a state in which the stent 41 is fully exposed to the outside in the second position, the stent 41 increases in diameter as a whole to widen the inner wall Cw of the constricted part C outward in the radial direction (see FIG. 4E). Subsequently, the distal end shaft 11 is drawn out toward the proximal end side so as to pass through the inside of the self-expanded stent 41 (see FIG. 4F), and the stent delivery system 1 from which the stent 41 is separated is removed out of the body. Thereby, the series of procedures for placing the stent 41 in the digestive tract K is completed.

As described above, since the stent delivery system 1 has the aforementioned configuration, widening of the constricted part C and placement of the stent 41 can be continuously performed, and the stent 41 can be smoothly placed in a body cavity such as the digestive tract K.

Note that the disclosed embodiments are not limited to the configurations of the aforementioned embodiments, but is stipulated by claims, and the disclosed embodiments are intended to include all modifications within the meaning and scope equivalent to those in claims.

For example, in the aforementioned embodiments, the stent delivery system 1 in which the outer peripheral edge of the proximal end of the distal end shaft 11 and the outer peripheral edge of the distal end of the outer shaft 51 coincide with each other at the first position, has been explained, but the stent delivery system may have any aspect as long as the outermost periphery of the outer shaft fits inside the outermost periphery of the spirally-arranged protruding portion in a frontal view.

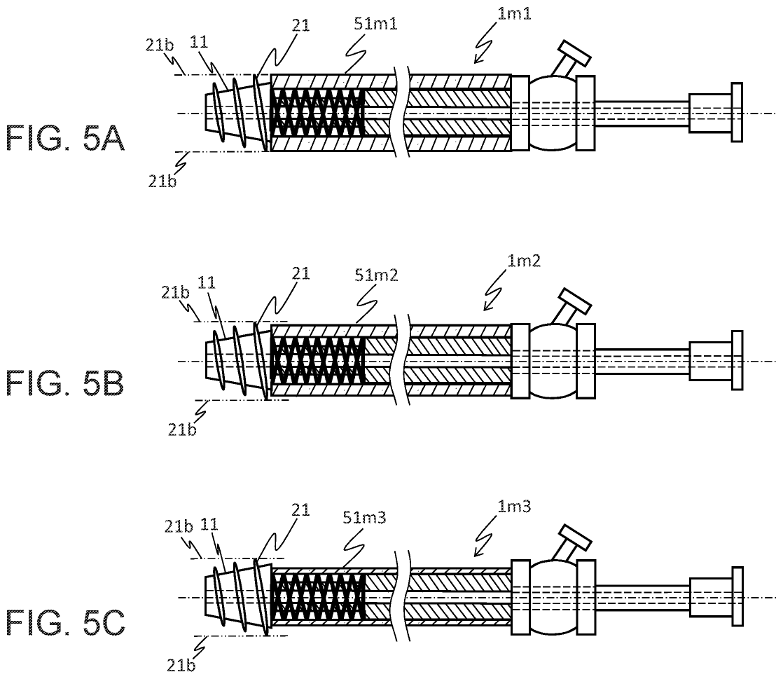

Examples of such a stent delivery system include a stent delivery system 1m1 in which an outermost periphery of an outer shaft 51m1 has the same shape as of the outermost periphery of the spirally-arranged protruding portion 21 in a frontal view (see FIG. 5A), a stent delivery system 1m2 in which an outermost periphery of an outer shaft 51m2 is located inside the outermost periphery of the spirally-arranged protruding portion 21 and outside the outer peripheral edge of the proximal end of the distal end shaft 11 (see FIG. 5B), a stent delivery system 1m3 in which an outermost periphery of an outer shaft 51m3 is located inside the outermost periphery of the spirally-arranged protruding portion 21 and inside the outer peripheral edge of the proximal end of the distal end shaft 11 (see FIG. 5C), and the like.

1. A stent delivery system comprising:
a distal end shaft comprising:
a distal end shaft through-hole extending along a longitudinal direction of the stent delivery system, and
a tapered portion having an outer diameter gradually increasing from a distal end of the tapered portion to a proximal end of the tapered portion;
a spirally-arranged protruding portion that is provided on an outer peripheral face of the tapered portion and has a gap between adjacent sections of the spirally-arranged protruding portion along the longitudinal direction;
an inner shaft that is connected to a proximal end of the distal end shaft and extends proximal to the distal end shaft in the longitudinal direction, the inner shaft comprising:
an inner shaft through-hole communicating with the distal end shaft through-hole;
a self-expanding stent that covers the inner shaft and is expandable and contractable in a radial direction of the inner shaft; and
an outer shaft that covers the inner shaft and is slidable along the longitudinal direction,
wherein:
the outer shaft is slidable between (i) a first position in which the stent is in a contracted state and is covered by the outer shaft, and (ii) a second position proximal to the stent in the longitudinal direction, and
an outermost periphery of the outer shaft fits inside an outermost periphery of the spirally-arranged protruding portion in a frontal view from a distal end side of the distal end shaft in the longitudinal direction.

2. The stent delivery system according to claim 1, wherein:
an outer diameter of the proximal end of the distal end shaft and an outer diameter of a distal end of the outer shaft substantially coincide with each other, and
the proximal end of the distal end shaft and the distal end of the outer shaft contact with each other such that an outer peripheral edge of the proximal end of the distal end shaft and an outer peripheral edge of the distal end of the outer shaft coincide with each other at the first position.
